SEQUENCE LISTING (7 sequences)

SEQ ID NO: 1 - DNA, 58 bp, Artificial Sequence
  Illumina Universal Adapter nucleic acid sequence
aatgatacggcgaccaccgagatctacactctttccctacacgacgctcttccgatct

SEQ ID NO: 2 - DNA, 33 bp, Artificial Sequence
  Illumina Index Adapter nucleic acid sequence
gatcggaagagcacacgtctgaactccagtcac

SEQ ID NO: 3 - DNA, 24 bp, Artificial Sequence
  Illumina Index Adapter nucleic acid sequence
atctcgtatgccgtcttctgcttg

SEQ ID NO: 4 - protein, 9 aa, Artificial Sequence
  neo-antigen amino acid sequence
ASLTHVDSL

SEQ ID NO: 5 - protein, 9 aa, Artificial Sequence
  neo-antigen amino acid sequence
SAYEKLYSL

SEQ ID NO: 6 - protein, 8 aa, Artificial Sequence
  neo-antigen amino acid sequence
ANFIFRQL

SEQ ID NO: 7 - protein, 9 aa, Artificial Sequence
  neo-antigen amino acid sequence
TIYVFPERL

(19) United States
(12) Patent Application Publication
(10) Pub. No.: US 2021/0315983 A1
(43) Pub. Date: Oct. 14, 2021

(54) VACCINATION WITH CANCER NEOANTIGENS

(71) Applicant: Yeda Research and Development Co. Ltd., Rehovot (IL)

(72) Inventors: Yardena SAMUELS, Rehovot (IL); Yochai WOLF, Rehovot (IL); Osnat BARTOK, Rehovot (IL)

(73) Assignee: Yeda Research and Development Co. Ltd., Rehovot (IL)

(21) Appl. No.: 17/092,386

(22) PCT Filed: May 14, 2019
(86) PCT No.: PCT/IL2019/050547

§ 371 Date: Nov. 9, 2020

(30) Foreign Application Priority Data
May 15, 2018 (IL) 259392

(51) Int. Cl. A61K 39/00; A61P 35/00; C12N 5/09; G01N 33/574

(52) U.S. Cl. CPC A61K 39/0011; A61P 35/00; C12N 5/0693; C12N 2503/00; A61K 2039/5152; A61K 2039/876; G01N 33/57492

(57) ABSTRACT

A method of identifying a neoantigen in a tumor of a subject is disclosed. The method comprises:

(a) culturing cells of the tumor under conditions which generate single cell clones of the tumor; and

(b) analyzing the single cell clones for expression of a neoantigen which is reactive with T cells from the subject, thereby identifying the neoantigen in a tumor of a subject.

### RELATED APPLICATIONS

This application is a Continuation of PCT Patent Application No. PCT/IL2019/050547 having international filing date of May 14, 2019, which claims the benefit of priority of Israel Patent Application No. 259392 filed on May 15, 2018. The contents of the above applications are all incorporated by reference as if fully set forth herein in their entirety.

### SEQUENCE LISTING STATEMENT

The ASCII file, entitled 84732Sequence Listing.txt, created on Nov. 9, 2020, comprising 1,765 bytes, submitted concurrently with the filing of this application is incorporated herein by reference.

### FIELD AND BACKGROUND OF THE INVENTION

The present invention, in some embodiments thereof, relates to a method of uncovering cancer neoantigens in tumor cells by generation of single cell clones. The single cell clones can be used in vaccinations for treating cancer in general, and more particularly, but not exclusively, for treating melanoma.

The concept that tumours express specific antigens that could render them naturally immunogenic with the provision of adequate immunostimulation was supported by the pioneering work of William B. Coley in the 1890s. Repeated injections of erysipelas, a bacterial toxin prepared from Streptococcus pyogenes, led to tumour regression in a patient with advanced sarcoma. This early work showed the potential for exogenously administered components to stimulate the immune response to achieve clinically evident tumour regression.

Hampered by the general lack of knowledge of tumour antigens, further progress in the development of cancer vaccines took more than a century. Initial cancer vaccines were developed from autologous tumour cells in the 1980s; one example is an autologous tumour cell—Mycobacterium Bovis bacillus Calmette Guérin (BCG) vaccine for patients with colorectal cancer, which showed modest clinical benefit in a small cohort of patients. In the early 1990s, melanoma-associated antigen 1 (MAGE1) was identified as the first human cancer antigen by screening of tumour-specific T cell clones with a tumour cDNA expression library. Human tumour antigens were also discovered through mass spectrometry-based or biochemical approaches used to identify sequences of peptides eluted from tumour-derived peptide—MHC complexes. Another antigen-identification approach used B cell-based cDNA expression cloning (SERER) with patient sera as an antibody source to screen tumour-derived cDNA expression libraries; this method identified the cancer—testis antigen 1 (CTAG1A; commonly known and referred to as NY-ESO-1).

In parallel with antigen discovery, there have been numerous efforts to break immune tolerance to tumour antigens and improve antigen delivery. The discovery of dendritic cells (DCs) in 1973 and recognition of their potent antigen-presenting capacities led to intense efforts towards DC vaccination. Other complex vaccine formats included whole tumour cell-based vaccines that are genetically modified to secrete granulocyte—macrophage colony-stimulating factor (GM-CSF), which were shown to mobilize CD4+ and CD8+ T cell responses against autologous tumours. Predicted immunogenic viral determinants in human papillomavirus (HPV)-driven carcinomas, administered as synthetic long peptides, also showed clinical benefit for patients with vulvar intraepithelial neoplasia. In 2010, the autologous DC-based prostate cancer vaccine Sipuleucel-T (Provenge; Dendreon) became the first human therapeutic cancer vaccine to be approved by the US Food and Drug Administration (FDA). Multiple clinical studies of vaccines targeting tumour-associated antigens have been carried out, such as with IMA901, a multipeptide vaccine for renal cell cancer, in 2012 and the GVAX pancreatic cancer vaccine in 2015. Polyvalent neoantigen-based vaccines have shown antitumour activity preclinically and have been tested in early human clinical trials.

WO2012159754A2 teaches vaccines, which are specific for a patient's tumor.

U.S. Pat. No. 6,207,147B1 and U.S. Pat. No. 6,277,368B1 teaches vaccines comprising cancer cells.

Additional background art includes Zhuting et al Nature reviews Immunology 18 p. 168-182 (2018).

### SUMMARY OF THE INVENTION

According to an aspect of some embodiments of the present invention there is provided a method of identifying a neoantigen in a tumor of a subject comprising:

(a) culturing cells of the tumor under conditions which generate single cell clones of the tumor; and

(b) analyzing the single cell clones for expression of a neoantigen, which is reactive with T cells from the subject, thereby identifying the neoantigen in a tumor of a subject.

According to an aspect of some embodiments of the present invention there is provided a vaccine comprising cells of a single tumor cell clone, the single tumor cell clone expressing at least one neoantigen, wherein a variant allele frequency (VAF) of the exonic mutations of the cells is greater than 0.25.

According to an aspect of some embodiments of the present invention there is provided a method of preparing a vaccine for treating a tumor of a subject comprising:

(a) culturing cells of the tumor under conditions which generate single cell clones of the tumor; and

(b) analyzing the single cell clones for expression of a neoantigen which is reactive with T cells from the subject; and

(c) preparing a vaccine, which comprises the neoantigen.

According to an aspect of some embodiments of the present invention there is provided a method of treating a cancer of a subject comprising:

(a) preparing a vaccine as described herein; and

(b) vaccinating the subject with the vaccine, thereby treating the cancer of the subject.

According to some embodiments of the invention, the cells of the tumor comprise cells of a solid tumor.

According to some embodiments of the invention, the cells of the tumor comprise melanocytes.

According to some embodiments of the invention, the culturing comprises passaging the cells of the tumor for no more than 20 passages.

According to some embodiments of the invention, the culturing comprises passaging the cells of the tumor for no more than 10 passages.

According to some embodiments of the invention, the vaccine comprises a single a single cell clone which expresses the neoantigen.

According to some embodiments of the invention, a variant allele frequency (VAF) of the exonic mutations of cells of the single cell clone is greater than 0.25.

According to some embodiments of the invention, a variant allele frequency (VAF) of the exonic mutations of cells of the single cell clone is greater than 0.65.

According to some embodiments of the invention, the vaccine comprises no more than ten single cell clones.

According to some embodiments of the invention, the vaccine comprises dendritic cells which present the neoantigen.

According to some embodiments of the invention, the tumor comprises cells of a solid tumor.

According to some embodiments of the invention, the cells of the tumor comprises melanoma cells.

According to some embodiments of the invention, the single tumor cell clone comprises primary cells.

According to some embodiments of the invention, the cells are viable.

According to some embodiments of the invention, the cells are irradiated.

According to some embodiments of the invention, the vaccine According to some embodiments of the invention, the further comprising an adjuvant.

According to some embodiments of the invention, the single tumor cell clone has been passaged for no more than 20 passages.

According to some embodiments of the invention, the tumor cell clone comprises melanocytes.

According to some embodiments of the invention, the vaccine is for use in treating a cancer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F illustrate that differential heterogeneity induces differential anti-tumor immunity. A) Scheme of experimental design for generating UVB irradiated cells and generating single cell clones derived from UVB irradiated cells. Cell lines are irradiated by UVB at dosage of 600 J/m2; from these irradiated cells, single cell clones are generated. B) Tumors grown in mice inoculated with UVB irradiated B2905 vs. clone 1 and 2, day 19. C) Tumors excised from UVB irradiated B2905 Vs. clone 2, day 19. D) In vivo growth of tumors in mice inoculated with single cell clone 2 (right panel). n=5, data is representative of two independent experiments. **p<0.01***p<0.001, two-way Annova followed by Bonferoni's post-hoc test with UVB irradiated B2905 derived tumors serve as control. E) In vivo growth of tumors derived from clone 1 in wild-type (full lines) and CD80/86−/−mice (dashed lines). N=4, **P<0.01, ***p<0.001, two-way Annova followed by Bonferoni's post-hoc test. F) In vivo growth of tumors derived from clone 2 in wild-type (full lines) Vs. CD80/86−/−mice (dashed lines). N=5-4, ***p<0.001, two-way Annova followed by Bonferoni's post-hoc.

FIGS. 2A-B illustrate in vivo tumor growth of B 16F10.9 derived cell lines.

A) In vivo tumor growth in mice inoculated with UVB irradiated B 16F10.9 (left) or B16 clone 1 (right) and B16 clone 2 (right panel). N=5, data is representative of two independent experiments. B) tumor day of onset clone 1 in WT vs. CD80/86^{-/-} mice. ***p<0.001, Mann-Whitney's U test, N=4-5.

FIGS. 3A-C. Detection of HLA-bound neoantigens in tumors with varying heterogeneity. A) FACS analysis of Splenic T cell co-cultured with BM-derived DCs incubated with the designated peptides. B) IFI44L>S tetramer stain of CD8+ TILs obtained from parental, UVB or clone 2 derived tumors at day 14 post inoculation. C) Summary of neo-peptide positive TILs as represented in B). N=3-6, p=0.0004, Kruskal-Wallis test.

FIGS. 4A-B. The degree of tumor heterogeneity dictates anti-tumor response in a quantitative manner proportional to the number of clones. A) In vivo tumor growth of tumors derived from UVB irradiated B2905 and various mixtures of single cell clones. Pie charts represent the degree of clonality in each mixture. B) Individual area under curve (AUC) for each mouse depicted in A. Pearson correlation is calculated for the various single cell clone mixtures.

FIGS. 5A-B. Phylogenetic tree analysis of the UVB-irradiated cell line. Reconstruction and visualization of clonal evolution in order to track tumor progression and intra-tumor heterogeneity. Copy number alterations and variant allele frequencies of somatic mutations were used to cluster the variants and infer the subclonal composition of the samples (SciClone). Subsequently, ClonEvol R-package was used for branch-based tree construction in order to graphically present clonal evolution and relationships between samples (parental cell line (A) and its derivative single cell clones (B)).

FIGS. 6A-B. Autologous single cell clone vaccination limits the growth of the parental UVB-irradiated line. In vivo tumor growth in mice inoculated with 6 different single cell clones derived from the UVB-irradiated B2905 line and one clone derived from a different parental clone (left; 5*10⁵ cells were inoculated via intradermal injection to the right lower flank) N=3-5 in each group; 38 days later, these mice and untreated, age-matched mice (black lines) were inoculated with the UVB-irradiated B2905 line (Right; 5*10⁵ cells, via intradermal injection to the left lower flank). Tumor volume was monitored every 2-3 days and measured with a caliper.

### DESCRIPTION OF SPECIFIC EMBODIMENTS OF THE INVENTION

The present invention, in some embodiments thereof, relates to a method of uncovering cancer neoantigens in tumor cells by generation of single cell clones. The single cell clones can be used in vaccinations for treating cancer in general, and more particularly, but not exclusively, for treating melanoma.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

It has recently been shown that immunotherapy strategies that enhance anti-tumor T-cell response, such as checkpoint inhibitors and adoptive T-cell therapy, exhibit remarkable clinical effects in a wide range of tumor types. However, many tumors do not respond to checkpoint inhibitors and the determinants of treatment efficacy remain largely unknown. Neo-antigens that arise as a consequence of somatic mutations within the tumor represent an attractive means to promote immune recognition in cancer. Indeed, high mutational and neo-antigen load in tumors has been associated with an enhanced response to immune checkpoint blockade therapy. Cutaneous melanoma, which is among the most highly mutated malignancies has the highest objective response rates to checkpoint blockade (˜60% upon combined CTLA4 & PD-1 blockade). There is a growing appreciation of the key role of T-cell mediated responses against neo-antigens in mediating responses to melanoma therapy.

While the current hypothesis in the immunotherapy field is that tumors with increased mutational loads present more neo-antigens and, thus, are more immunogenic, tumors containing equally high mutational loads exhibit a variable immune response.

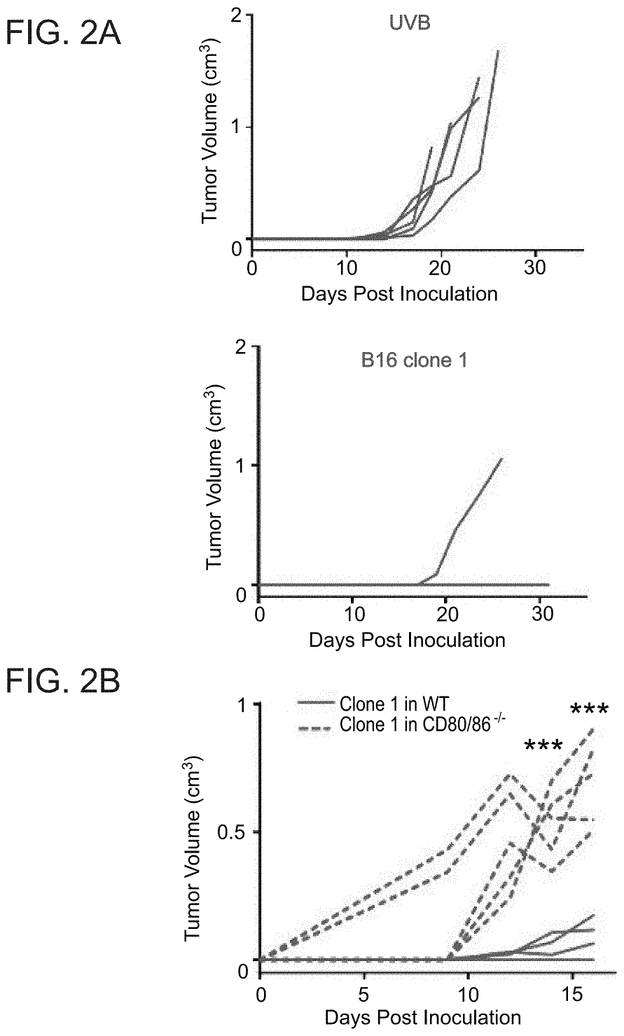

The present inventors explored the role of intra-tumor heterogeneity (ITH) on tumor rejection by establishing a melanoma mouse model and inducing ultra-violet B (UVB)-derived mutations that increase both ITH and the mutational load. This induction gave rise to highly aggressive tumors due to decreased cytotoxic activity of tumor infiltrating Lymphocytes (TILs). In contrast, single cell-derived melanoma clones that have reduced ITH were rejected (FIGS. 2A-B). This tumor rejection is accompanied by increased TIL reactivity, increased infiltration into the tumor core, and a distinct spectrum of TCR repertoire and HLA bound neo-antigens.

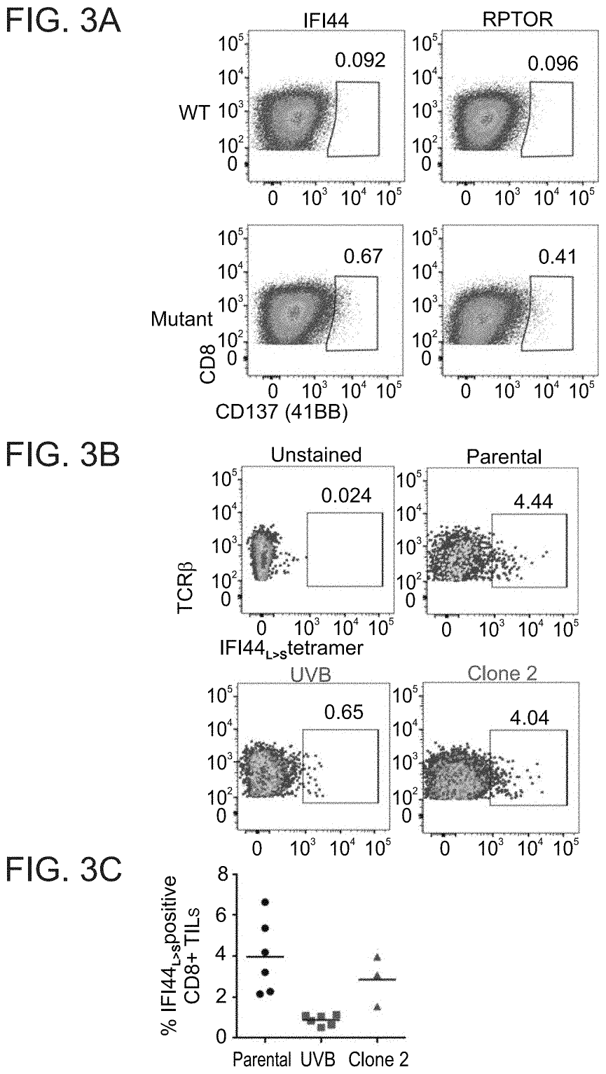

Whilst further reducing the present invention to practice, the present inventors have now uncovered that generating single cell clones enables detection of immunodominant neoantigens that could not be detected in the original patient derived cell line due to their low frequency (FIG. 3A).

Thus the present inventors propose that generation of single cell clones would allow for better detection of neoantigens which can be further used in vaccination (either for dendritic cell vaccination, e.g. by mRNA or peptide loading).

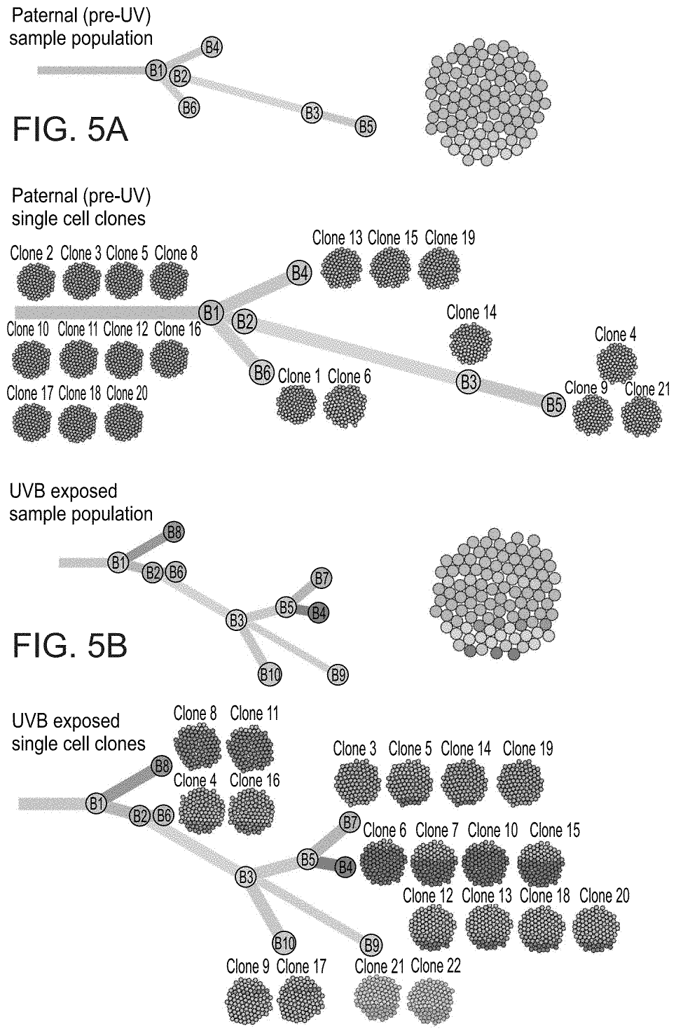

The present inventors further showed that when several clones are combined in ascending numbers, anti-tumor immunity is dampened in a manner proportional to the amount of clones in the mix (FIGS. 5A-B). Thus, the present inventors propose direct vaccination of the single cell clones themselves for the treatment of cancer.

Thus, according to a first aspect of the present invention, there is provided a method of identifying a neoantigen in a tumor of a subject comprising:

(a) culturing cells of the tumor under conditions which generate single cell clones of said tumor; and

(b) analyzing said single cell clones for expression of a neoantigen, which is reactive with T cells from said subject, thereby identifying the neoantigen in a tumor of a subject.

As used herein the term “neoantigen” is an epitope that has at least one alteration that makes it distinct from the corresponding wild-type, parental antigen, e.g., via mutation in a tumor cell or post-translational modification specific to a tumor cell. A neoantigen can include a polypeptide sequence or a nucleotide sequence. A mutation can include a frameshift or nonframeshift indel, missense or nonsense substitution, splice site alteration, genomic rearrangement or gene fusion, or any genomic or expression alteration giving rise to a neoORF. A mutation can also include a splice variant. Post-translational modifications specific to a tumor cell can include aberrant phosphorylation. Post-translational modifications specific to a tumor cell can also include a proteasome-generated spliced antigen.

In one embodiment, the neoantigen is a short peptide that is bound to a class I or II MHC receptor thus forming a ternary complex that can be recognized by a T-cell bearing a matching T-cell receptor binding to the MHC/peptide complex with appropriate affinity. Peptides binding to MHC class I molecules are typically about 8-14 amino acids in length. T-cell epitopes that bind to MHC class II molecules are typically about 12-30 amino acids in length. In the case of peptides that bind to MHC class II molecules, the same peptide and corresponding T cell epitope may share a common core segment, but differ in the overall length due to flanking sequences of differing lengths upstream of the amino-terminus of the core sequence and downstream of its carboxy terminus, respectively. A T-cell epitope may be classified as an antigen if it elicits an immune response.

Proteins from which the neoantigens are derived comprise cancer-associated modifications. Exemplary modifications include, but are not limited to cancer associated mutations and cancer-associated phosphorylation patterns.

The term “mutation” refers to a change of or difference in the nucleic acid sequence (nucleotide substitution, addition or deletion) compared to a reference. A “somatic mutation” can occur in any of the cells of the body except the germ cells (sperm and egg) and therefore are not passed on to children. These alterations can (but do not always) cause cancer or other diseases. Preferably, a mutation is a non-synonymous mutation. The term “non-synonymous mutation” refers to a mutation, preferably a nucleotide substitution, which does result in an amino acid change such as an amino acid substitution in the translation product.

According to the invention, the term “mutation” includes point mutations, Indels, fusions, chromothripsis and RNA edits.

According to the invention, the term “Indel” describes a special mutation class, defined as a mutation resulting in a colocalized insertion and deletion and a net gain or loss in nucleotides. In coding regions of the genome, unless the length of an indel is a multiple of 3, they produce a frameshift mutation. Indels can be contrasted with a point mutation; where an Indel inserts and deletes nucleotides from a sequence, a point mutation is a form of substitution that replaces one of the nucleotides. In one embodiment, the indel is a frameshift deletion mutation. In another embodiment, the indel is a frameshift insertion mutation.

Fusions can generate hybrid genes formed from two previously separate genes. It can occur as the result of a translocation, interstitial deletion, or chromosomal inversion. Often, fusion genes are oncogenes. Oncogenic fusion genes may lead to a gene product with a new or different function from the two fusion partners. Alternatively, a proto-oncogene is fused to a strong promoter, and thereby the oncogenic function is set to function by an upregulation caused by the strong promoter of the upstream fusion partner. Oncogenic fusion transcripts may also be caused by trans-splicing or read-through events.

According to the invention, the term “chromothripsis” refers to a genetic phenomenon by which specific regions of the genome are shattered and then stitched together via a single devastating event.

According to the invention, the term “RNA edit” or “RNA editing” refers to molecular processes in which the information content in an RNA molecule is altered through a chemical change in the base makeup. RNA editing includes nucleoside modifications such as cytidine (C) to uridine (U) and adenosine (A) to inosine (I) deaminations, as well as non-templated nucleotide additions and insertions. RNA editing in mRNAs effectively alters the amino acid sequence of the encoded protein so that it differs from that predicted by the genomic DNA sequence.

Preferably, the mutations are non-synonymous mutations, preferably non-synonymous mutations of proteins expressed in a tumor or cancer cell.

In a particular embodiment, the protein, which expresses a cancer-related modification pattern, is expressed in melanoma cells, lung cancer cells, renal cancer cells or Head and neck squamous carcinoma cells.

Preferably, the protein, which expresses a cancer-related modification pattern, is expressed in melanoma cells.

Examples of proteins which may express cancer related modification patterns include, but are not limited to kallikrein 4, papillomavirus binding factor (PBF), preferentially expressed antigen of melanoma (PRAME), Wilms' tumor-1 (WT1), Hydroxysteroid Dehydrogenase Like 1 (HSDL1), mesothelin, cancer testis antigen (NY-ESO-1), carcinoembryonic antigen (CEA), p53, human epidermal growth factor receptor 2/neuro receptor tyrosine kinase (Her2/Neu), carcinoma-associated epithelial cell adhesion molecule EpCAM), ovarian and uterine carcinoma antigen (CA125), folate receptor a, sperm protein 17, tumor-associated differentially expressed gene-12 (TADG-12), mucin-16 (MUC-16), L1 cell adhesion molecule (L1CAM), mannan-MUC-1, Human endogenous retrovirus K (HERV-K-MEL), Kita-kyushu lung cancer antigen-1 (KK-LC-1), human cancer/testis antigen (KM-HN-1), cancer testis antigen (LAGE-1), melanoma antigen-A1 (MAGE-A1), Sperm surface zona pellucida binding protein (Sp17), Synovial Sarcoma, X Breakpoint 4 (SSX-4), Transient axonal glycoprotein-1 (TAG-1), Transient axonal glycoprotein-2 (TAG-2), Enabled Homolog (ENAH), mammoglobin-A, NY-BR-1, Breast Cancer Antigen, (BAGE-1), B melanoma antigen, melanoma antigen-A1 (MAGE-A1), melanoma antigen-A2 (MAGE-A2), mucin k, synovial sarcoma, X breakpoint 2 (SSX-2), Taxol-resistance-associated gene-3 (TRAG-3), Avian Myelocytomatosis Viral Oncogene (c-myc), cyclin B1, mucin 1 (MUC1), p62, survivin, lymphocyte common antigen (CD45), Dickkopf WNT Signaling Pathway Inhibitor 1 (DKK1), telomerase, Kirsten rat sarcoma viral oncogene homolog (K-ras), G250, intestinal carboxyl esterase, alpha-fetoprotein, Macrophage Colony-Stimulating Factor (M-CSF), Prostate-specific membrane antigen (PSMA), caspase 5 (CASP-5), Cytochrome C Oxidase Assembly Factor 1 Homolog (COA-1), O-linked (3-N-acetylglucosamine transferase (OGT), Osteosarcoma Amplified 9, Endoplasmic Reticulum Lectin (OS-9), Transforming Growth Factor Beta Receptor 2 (TGF-betaRll), murine leukemia glycoprotein 70 (gp70), Calcitonin Related Polypeptide Alpha (CALCA), Programmed cell death 1 ligand 1 (CD274), Mouse Double Minute 2Homolog (mdm-2), alpha-actinin-4, elongation factor 2, Malic Enzyme 1 (ME1), Nuclear Transcription Factor Y Subunit C (NFYC), G Antigen 1,3 (GAGE-1,3), melanoma antigen-A6 (MAGE-A6), cancer testis antigen XAGE-1b, six transmembrane epithelial antigen of the prostate 1 (STEAP1), PAP, prostate specific antigen (PSA), Fibroblast Growth Factor 5 (FGF5), heat shock protein hsp70-2, melanoma antigen-A9 (MAGE-A9), Arg-specific ADP-ribosyltransferase family C (ARTC1), B-Raf Proto-Oncogene (B-RAF), Serine/Threonine Kinase, beta-catenin, Cell Division Cycle 27 homolog (Cdc27), cyclin dependent kinase 4 (CDK4), cyclin dependent kinase 12 (CDK12), Cyclin Dependent Kinase Inhibitor 2A (CDKN2A), Casein Kinase 1 Alpha 1 (CSNK1A1), Fibronectin 1 (FN1), Growth Arrest Specific 7 (GAS7), Glycoprotein nonmetastatic melanoma protein B (GPNMB), HAUS Augmin Like Complex Subunit 3 (HAUS3), LDLR-fucosyltransferase, Melanoma Antigen Recognized By T-Cells 2 (MART2), myostatin (MSTN), Melanoma Associated Antigen (Mutated) 1 (MUM-1-2-3), Poly(A) polymerase gamma (neo-PAP), myosin class I, Protein phosphatase 1 regulatory subunit 3B (PPP1R3B), Peroxiredoxin-5 (PRDXS), Receptor-type tyrosine-protein phosphatase kappa (PTPRK), Transforming protein N-Ras (N-ras), retinoblastoma-associated factor 600 (RBAF600), sirtuin-2 (SIRT2), SNRPD1, triosephosphate isomerase, Ocular Albinism Type 1 Protein (OA1), member RAS oncogene family (RAB38), Tyrosinase related protein 1-2 (TRP-1-2), Melanoma Antigen Gp75 (gp75), tyrosinase, Melan-A (MART-1), Glycoprotein 100 melanoma antigen (gp100), N-acetylglucosaminyltransferase V gene (GnTVf), Lymphocyte Antigen 6 Complex Locus K (LY6K), melanoma antigen-A10 (MAGE-A10), melanoma antigen-A12 (MAGE-A12), melanoma antigen-C2 (MAGE-C2), melanoma antigen NA88-A, Taxol-resistant-associated protein 3 (TRAG-3), BDZ binding kinase (pbk), caspase 8 (CASP-8), sarcoma antigen 1 (SAGE), Breakpoint Cluster Region-Abelson oncogene (BCR-ABL), fusion protein in leukemia, dek-can, Elongation Factor Tu GTP Binding Domain Containing 2 (EFTUD2), ETS Variant gene 6/acute myeloid leukemia fusion protein (ETV6-AML1), FMS-like tyrosine kinase-3 internal tandem duplications (FLT3-ITD), cyclin-A1, Fibronectin Type III Domain Containing 3B (FDNC3B,) promyelocytic leukemia/retinoic acid receptor alpha fusion protein (pml-RARalpha), melanoma antigen-C1 (MAGE-C1), membrane protein alternative spliced isoform (D393-CD20), melanoma antigen-A4 (MAGE-A4), or melanoma antigen-A3 (MAGE-A3).

Additional examples of proteins that may express cancer related modification patterns are known in the art and are described, for example, in Reuschenbach et al., Cancer Immunol. Immunother. 58:1535-1544 (2009); Parmiani et al., J. Nat. Cancer Inst. 94:805-818 (2002); Zarour et al., Cancer Medicine. (2003); Bright et al., Hum. Vaccin. Immunother. 10:3297-3305 (2014); Wurz et al., Ther. Adv. Med. Oncol. 8:4-31 (2016); Criscitiello, Breast Care 7:262-266 (2012); Chester et al., J. Immunother. Cancer 3:7 (2015); Li et al., Mol. Med. Report 1:589-594 (2008); Liu et al., J. Hematol. Oncol. 3:7 (2010); Bertino et al., Biomed. Res. Int. 731469 (2015); and Suri et al., World J. Gastrointest. Oncol. 7:492-502 (2015).

In one embodiment, the mutations are cancer specific somatic mutations.

As used herein, the term “cell(s) of a tumor” denotes a cell which is located within a tumor or a tumor environment (e.g. site of metastasis). In one embodiment, the cell is malignant (i.e., capable of metastasis and the mediation of disease). In another embodiment, the cell is of a solid tumor (i.e. does not include Tumor Infiltrating Lymphocytes (TILs), leucocytes, macrophages, and/or other cells of the immune system). In another embodiment, the cell of the tumor is not a stromal cell, and/or fibroblast.

The subject (e.g., patient) and the tumors which are analyzed in accordance with the present disclosure may be of any mammalian species (e.g., human, or primate, canine, feline, bovine, ovine, equine, porcine, rodent species (e.g., murine), etc.). The disclosure particularly concerns the analysis of human tumor cells.

The tumor cells of relevance to the present disclosure include, but are not limited to, tumor cells of cancers, including but not limited to adrenocortical carcinoma, hereditary; bladder cancer; breast cancer; breast cancer, ductal; breast cancer, invasive intraductal; breast cancer, sporadic; breast cancer, susceptibility to; breast cancer, type 4; breast cancer, type 4; breast cancer-1; breast cancer-3; breast-ovarian cancer; Burkitt's lymphoma; cervical carcinoma; colorectal adenoma; colorectal cancer; colorectal cancer, hereditary nonpolyposis, type 1; colorectal cancer, hereditary nonpolyposis, type 2; colorectal cancer, hereditary nonpolyposis, type 3; colorectal cancer, hereditary nonpolyposis, type 6; colorectal cancer, hereditary nonpolyposis, type 7; dermatofibrosarcoma protuberans; endometrial carcinoma; esophageal cancer; gastric cancer, fibrosarcoma, glioblastoma multiforme; glomus tumors, multiple; hepatoblastoma; hepatocellular cancer; hepatocellular carcinoma; leukemia, acute lymphoblastic; leukemia, acute myeloid; leukemia, acute myeloid, with eosinophilia; leukemia, acute nonlymphocytic; leukemia, chronic myeloid; Li-Fraumeni syndrome; liposarcoma, lung cancer; lung cancer, small cell; lymphoma, non-Hodgkin's; lynch cancer family syndrome II; male germ cell tumor; mast cell leukemia; medullary thyroid; medulloblastoma; melanoma, meningioma; multiple endocrine neoplasia; myeloid malignancy, predisposition to; myxosarcoma, neuroblastoma; osteosarcoma; ovarian cancer; ovarian cancer, serous; ovarian carcinoma; ovarian sex cord tumors; pancreatic cancer; pancreatic endocrine tumors; paraganglioma, familial nonchromaffin; pilomatricoma; pituitary tumor, invasive; prostate adenocarcinoma; prostate cancer; renal cell carcinoma, papillary, familial and sporadic; retinoblastoma; rhabdoid predisposition syndrome, familial; rhabdoid tumors; rhabdomyosarcoma; small-cell cancer of lung; soft tissue sarcoma, squamous cell carcinoma, head and neck; T-cell acute lymphoblastic leukemia; Turcot syndrome with glioblastoma; tylosis with esophageal cancer; uterine cervix carcinoma, Wilms' tumor, type 2; and Wilms' tumor, type 1, etc.

According to a particular embodiment, the tumor cell is a melanoma cell (e.g. melanocyte).

Tumor cells are generally sampled by a surgical procedure, including but not limited to biopsy, or surgical resection or debulking. Solid tumors can be dissociated into separate cells (i.e. single cell suspension) by physical manipulation optionally combined with enzymatic treatment with such enzymes as Hyaluronidase DNAase, Collagenase, Trypsin, Dispase and Neuraminidase and the like. The cells may then be transferred into fresh physiological or growth medium. Cells may be stored until further use, for example, by freezing in liquid nitrogen.

To generate single cell clones, a single cell is cultured in a single well (e.g. 96 well plate) until a clone is generated (e.g. about 1 weeks-3 weeks, e.g. about two weeks). Single cell clones are then visible (e.g. using a microscopy). The clones are then picked and expanded. Preferably the clones are not passaged for more than 10 passages, 11 passages, 12 passages, 13 passages, 14 passages, 15 passages, 16 passages, 17 passages, 18 passages, 19 passages or 20 passages.

According to this aspect of the present invention a single cell clone is defined as one wherein the exonic mutations of each of the cells of the clone have a variant allele frequency (VAF) greater than 0.25.

According to this aspect of the present invention a single cell clone is defined as one wherein the exonic mutations of each of the cells of the clone have a variant allele frequency (VAF) greater than 0.35.

According to this aspect of the present invention a single cell clone is defined as one wherein the exonic mutations of each of the cells of the clone have a variant allele frequency (VAF) greater than 0.45.

According to this aspect of the present invention a single cell clone is defined as one wherein the exonic mutations of each of the cells of the clone have a variant allele frequency (VAF) greater than 0.55.

According to this aspect of the present invention a single cell clone is defined as one wherein the exonic mutations of each of the cells of the clone have a variant allele frequency (VAF) greater than 0.65.

Analysis of VAF is described in Williams, M. J., Werner, B., Barnes, C. P., Graham, T. A. & Sottoriva, A. Identification of neutral tumor evolution across cancer types. Nature genetics 48, 238-244 (2016), the contents of which is incorporated herein by reference.

Any suitable sequencing method can be used to determine the VAF, Next Generation Sequencing (NGS) technologies being preferred. Third Generation Sequencing methods might substitute for the NGS technology in the future to speed up the sequencing step of the method. For clarification purposes: the terms “Next Generation Sequencing” or “NGS” in the context of the present invention mean all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks, preferably within 1-7 days or most preferably within less than 24 hours and allow, in principle, single cell sequencing approaches. Multiple NGS platforms which are commercially available or which are mentioned in the literature can be used in the context of the present invention e.g. those described in detail in Zhang et al. 2011: The impact of next-generation sequencing on genomics. J. Genet Genomics 38 (3), 95-109; or in Voelkerding et al. 2009: Next generation sequencing: From basic research to diagnostics. Clinical chemistry 55, 641-658. Non-limiting examples of such NGS technologies/platforms are [0164] 1) The sequencing-by-synthesis technology known as pyrosequencing implemented e.g. in the GS-FLX 454 Genome Sequencer™ of Roche-associated company 454 Life Sciences (Branford, Conn.), first described in Ronaghi et al. 1998: A sequencing method based on real-time pyrophosphate”. Science 281 (5375), 363-365. This technology uses an emulsion PCR in which single-stranded DNA binding beads are encapsulated by vigorous vortexing into aqueous micelles containing PCR reactants surrounded by oil for emulsion PCR amplification. During the pyrosequencing process, light emitted from phosphate molecules during nucleotide incorporation is recorded as the polymerase synthesizes the DNA strand. [0165] 2) The sequencing-by-synthesis approaches developed by Solexa (now part of Illumina Inc., San Diego, Calif.) which is based on reversible dye-terminators and implemented e.g. in the Illumina/Solexa Genome Analyzer™ and in the Illumina HiSeq 2000 Genome Analyze™. In this technology, all four nucleotides are added simultaneously into oligo-primed cluster fragments in flow-cell channels along with DNA polymerase. Bridge amplification extends cluster strands with all four fluorescently labeled nucleotides for sequencing. [0166] 3) Sequencing-by-ligation approaches, e.g. implemented in the SOLid™ platform of Applied Biosystems (now Life Technologies Corporation, Carlsbad, Calif.). In this technology, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. As a second example, he Polonator™ G.007 platform of Dover Systems (Salem, N. H.) also employs a sequencing-by-ligation approach by using a randomly arrayed, bead-based, emulsion PCR to amplify DNA fragments for parallel sequencing. [0167] 4) Single-molecule sequencing technologies such as e.g. implemented in the PacBio RS system of Pacific Biosciences (Menlo Park, Calif.) or in the HeliScope™ platform of Helicos Biosciences (Cambridge, Mass.). The distinct characteristic of this technology is its ability to sequence single DNA or RNA molecules without amplification, defined as Single-Molecule Real Time (SMRT) DNA sequencing. For example, HeliScope uses a highly sensitive fluorescence detection system to directly detect each nucleotide as it is synthesized. A similar approach based on fluorescence resonance energy transfer (FRET) has been developed from Visigen Biotechnology (Houston, Tex.). Other fluorescence-based single-molecule techniques are from U.S. Genomics (GeneEngine™.) and Genovoxx (AnyGene™). [0168] 5) Nano-technologies for single-molecule sequencing in which various nanostructures are used which are e.g. arranged on a chip to monitor the movement of a polymerase molecule on a single strand during replication. Non-limiting examples for approaches based on nano-technologies are the GridON™ platform of Oxford Nanopore Technologies (Oxford, UK), the hybridization-assisted nano-pore sequencing (HANS™TM^{T}) platforms developed by Nabsys (Providence, R.I.), and the proprietary ligase-based DNA sequencing platform with DNA nanoball (DNB) technology called combinatorial probe-anchor ligation (cPAL™) [0169] 6) Electron microscopy based technologies for single-molecule sequencing, e.g. those developed by LightSpeed Genomics (Sunnyvale, Calif.) and Halcyon Molecular (Redwood City, Calif.) [0170] 7) Ion semiconductor sequencing which is based on the detection of hydrogen ions that are released during the polymerisation of DNA. For example, Ion Torrent Systems (San Francisco, Calif.) uses a high-density array of micro-machined wells to perform this biochemical process in a massively parallel way. Each well holds a different DNA template. Beneath the wells is an ion-sensitive layer and beneath that a proprietary Ion sensor.

Preferably, DNA and RNA preparations serve as starting material for NGS. Such nucleic acids can be easily obtained from samples such as biological material, e.g. from fresh, flash-frozen or formalin-fixed paraffin embedded tumor tissues (FFPE) or from freshly isolated cells or from CTCs which are present in the peripheral blood of patients. Normal non-mutated genomic DNA or RNA can be extracted from normal, somatic tissue, however germline cells are preferred in the context of the present invention. Germline DNA or RNA may be extracted from peripheral blood mononuclear cells (PBMCs) in patients with non-hematological malignancies. Although nucleic acids extracted from FFPE tissues or freshly isolated single cells are highly fragmented, they are suitable for NGS applications.

Several targeted NGS methods for exome sequencing are described in the literature (for review see e.g. Teer and Mullikin 2010: Human Mol Genet 19 (2), R145-51), all of which can be used in conjunction with the present invention. Many of these methods (described e.g. as genome capture, genome partitioning, genome enrichment etc.) use hybridization techniques and include array-based (e.g. Hodges et al. 2007: Nat. Genet. 39, 1522-1527) and liquid-based (e.g. Choi et al. 2009: Proc. Natl. Acad. Sci USA 106, 19096-19101) hybridization approaches. Commercial kits for DNA sample preparation and subsequent exome capture are also available: for example, Illumina Inc. (San Diego, Calif.) offers the TruSee™. DNA Sample Preparation Kit and the Exome Enrichment Kit TruSeq™ Exome Enrichment Kit.

The generated single cell clones can optionally be analyzed (e.g. by whole exome sequencing (WES) so as to infer a phylogenetic tree. In this way, single cell clones can be selected that harbor clonal mutations, shared by all cells of the tumor, which are found in the stem of the tree. According to another embodiment, a mixture of single cell clones can be selected which are found in different branches of the phylogenetic tree.

The generated single cell clones are then analyzed for expression of at least one neoantigen.

This entails purifying MHC molecules from the single cell clones (e.g. by immunoaffinity) and then analyzing the MHC-eluted peptides using any of the following methods: thin layer chromatography, electrophoresis, in particular capillary electrophoresis, solid phase extraction (CSPE), reverse-phase high performance liquid chromatography, amino-acid analysis after acid hydrolysis and by fast atom bombardment (FAB) mass spectrometric analysis, as well as MALDI and ESI-Q-TOF mass spectrometric analysis.

In a particular embodiment, the amino acid sequence of the peptides displayed on the cells surface is determined using liquid chromatography and tandem mass spectrometry (LC—MS/MS) and/or HPLC.

The sequence of the peptides (e.g. derived from the MS spectra) may then be queried against a proteome dataset (e.g. human proteome dataset), to which is added the peptides inferred from the mutations identified by sequencing of the tumor cells (e.g. by whole exome sequencing (WES)), as further described herein above.

As mentioned the neoantigen which is uncovered is reactive with T cells.

Analyzing Reactivity of Neoantigen

Specific activation of CD4+ or CD8+ T cells may be detected in a variety of ways. Methods for detecting specific T cell activation include detecting the proliferation of T cells, the production of cytokines (e.g., lymphokines), or the generation of cytolytic activity. For CD4+ T cells, a preferred method for detecting specific T cell activation is the detection of the proliferation of T cells. For CD8+ T cells, a preferred method for detecting specific T cell activation is the detection of the generation of cytolytic activity.

According to a particular embodiment, in order to determine the reactivity of the peptides, an ELISPOT assay may be carried out, where the CD8+ CTL response, which can be assessed by measuring IFN-gamma production by antigen-specific effector cells, is quantitated by measuring the number of Spot Forming Units (SFU) under a stereomicroscope (Rininsland et al., (2000) J Immunol Methods: 240(1-2):143-155.). In this assay, antigen-presenting cells (APC) are immobilized on the plastic surface of a micro titer well, and effector T cells are added at various effector:target ratios. Antigen presenting cells are preferably B cells or dendritic cells. The binding of APC's by antigen-specific effector cells triggers the production of cytokines including IFN-gamma by the effector cells (Murali-Krishna et al., (1998) Adv Exp Med Biol.: 452:123-142). In one embodiment, subject-specific T cells are used in the ELISPOT assay. The amount of soluble IFNγ secreted from the TILs may also be measured by ELISA assay (e.g. Biolegend).

Another method for determining the reactivity of the peptides is by direct determination of cell lysis as measured by the classical assay for CTL activity namely the chromium release assay (Walker et al., (1987) Nature: 328:345-348; Scheibenbogen et al., (2000) J Immunol Methods: 244(1-2):81-89.). In one embodiment subject-specific T cells are used in this assay. Effector Cytotoxic T Lymphocytes (CTL) bind targets bearing antigenic peptide on Class I MHC and signal the targets to undergo apoptosis. If the targets are labeled with ⁵¹Chromium before the CTL are added, the amount of ⁵¹Cr released into the supernatant is proportional to the number of targets killed. Antigen-specific lysis is calculated by comparing lysis of target cells expressing disease or control antigens in the presence or absence of patient effector cells, and is usually expressed as the %-specific lysis. Percent specific cytotoxicity is calculated by (specific release-spontaneous release)/(maximum release-spontaneous release) and may be 20%-85% for a positive assay. Percent specific cytotoxicity is usually determined at several ratios of effector (CTL) to target cells (E:T). Additionally, the standard lytic assay is qualitative and must rely on a limiting dilution analysis (LDA) for quantitative results, and the LDA frequently underestimates the true level of CTL response. Although CTL can each kill many targets in vivo, in vitro this assay requires numbers of CTL equal to or greater than the number of targets for detectable killing. In one embodiment CTL responses are measured by the chromium release assay, monitoring the ability of T cells (Effector cells) to lyse radiolabelled HLA matched “target cells” that express the appropriate antigen-MHC complex.

Once a neoantigen has been identified and its reactivity confirmed, it can be used to generate a vaccine.

The efficacy of a vaccine can be analyzed prior to administration by measuring Delayed-Type Hypersensitivity (DTH) diameter. In one embodiment, a positive response is set at a measurement of >10 mm.

As used herein, the term “vaccine” refers to a pharmaceutical preparation (pharmaceutical composition) or product that upon administration induces an immune response, in particular a cellular immune response, which recognizes and attacks a pathogen or a diseased cell such as a cancer cell.

A vaccine may be used for the prevention or treatment of a disease such as cancer (e.g. melanoma). The term “personalized cancer vaccine” or “individualized cancer vaccine” concerns a particular cancer patient and means that a cancer vaccine is adapted to the needs or special circumstances of an individual cancer patient.

In one embodiment, the vaccine comprises a peptide identified as being immunogenic and expressed in tumor cells of the subject. In another embodiment, the vaccine comprises a nucleic acid, preferably RNA, encoding said peptide or polypeptide.

The cancer vaccines provided according to the invention when administered to a patient provide one or more epitopes suitable for stimulating, priming and/or expanding T cells specific for the patient's tumor. The T cells are preferably directed against cells expressing antigens from which the T cell epitopes are derived. Thus, the vaccines described herein are preferably capable of inducing or promoting a cellular response, preferably cytotoxic T cell activity, against a cancer disease characterized by presentation of one or more tumor-associated neoantigens with class I MHC. Since a vaccine provided according to the present invention will target cancer specific mutations it will be specific for the patient's tumor.

The vaccine can comprise one or more neoantigens identified according to the methods described herein, such as 2 or more, 5 or more, 10 or more, 15 or more, 20 or more, 25 or more, 30 or more and preferably up to 60, up to 55, up to 50, up to 45, up to 40, up to 35 or up to 30 T cell epitopes.

In one embodiment, the vaccine comprises neoantigens of a single cell clone and does not comprise neoantigens that are not present on the single cell clone.

The present invention further contemplates vaccines of antigen presenting cells which are loaded with the neoantigens that are identified according to the methods described herein.

Antigen presenting cells (APC) are cells which present peptide fragments of protein antigens in association with MHC molecules on their cell surface. Some APCs may activate antigen specific T cells.

According to a particular embodiment, the APCs used in the vaccine of the present invention expresses MHC class I and MHC class II molecules. Preferably, the APC can also stimulate CD4+ helper T cells as well as cytotoxic T cells.

Examples of APCs include, but are not limited to dendritic macrophages, Langerhans cells and B cells.

Dendritic cells (DCs) are leukocyte populations that present antigens captured in peripheral tissues to T cells via both MHC class II and I antigen presentation pathways. It is well known that dendritic cells are potent inducers of immune responses and the activation of these cells is a critical step for the induction of antitumoral immunity. Dendritic cells are conveniently categorized as “immature” and “mature” cells, which can be used as a simple way to discriminate between two well characterized phenotypes. However, this nomenclature should not be construed to exclude all possible intermediate stages of differentiation. Immature dendritic cells are characterized as antigen presenting cells with a high capacity for antigen uptake and processing, which correlates with the high expression of Fc.gamma. receptor and mannose receptor. The mature phenotype is typically characterized by a lower expression of these markers, but a high expression of cell surface molecules responsible for T cell activation such as class I and class II MHC, adhesion molecules (e.g. CD54 and CD11) and costimulatory molecules (e.g., CD40, CD80, CD86 and 4-1 BB). Dendritic cell maturation is referred to as the status of dendritic cell activation at which such antigen-presenting dendritic cells lead to T cell priming, while presentation by immature dendritic cells results in tolerance. Dendritic cell maturation is chiefly caused by biomolecules with microbial features detected by innate receptors (bacterial DNA, viral RNA, endotoxin, etc.), pro-inflammatory cytokines (TNF, IL-1, IFNs), ligation of CD40 on the dendritic cell surface by CD40L, and substances released from cells undergoing stressful cell death. The dendritic cells can be derived by culturing bone marrow cells in vitro with cytokines, such as granulocyte-macrophage colony-stimulating factor (GM-CSF) and tumor necrosis factor alpha.

In one embodiment, the vaccine comprises dendritic cells derived from a patient's own cells. In one protocol, a large amount of peripheral blood mononuclear cells (PBMCs) are harvested from the patient via an invasive leukapheresis process. Monocytes are then isolated from PBMCs and differentiated into DCs. These monocyte-derived DCs (moDCs) are loaded with tumor antigens, matured and injected back to the patient.

Currently, several antigen loading approaches have been used in DC vaccine production. Protein- or tumor lysate-loading provides the possibility to present multiple antigenic epitopes without being restricted by a subject's MHC haplotype.

Peptide-pulsing is a simple approach to load DCs with tumor antigen for presentation to CD8+ T cells, in which the MHC-restricted tumor antigenic peptides bind directly to the MHC class I molecule without going through the antigen processing pathways.

Nucleic acid-based antigen loading approach may extend tumor antigen presentation duration in DCs. In this approach, tumor antigen-coding DNA or RNA are delivered into DCs and the expression of these tumor antigen-coding nucleic acids may provide an endogenous supply of cytosolic tumor antigens that incline to be presented via endogenous pathway. The antigen presentation efficiency using such approach depends largely on high-level transgene expression in DCs. For DNA-based antigen loading, viral vectors tend to be used. For RNA-based antigen loading, tumor antigen-coding RNA can be delivered via electroporation into the DC cytoplasm, where the RNA is translated to produce tumor antigens.

The present invention further contemplates vaccines comprising single cell clones of the tumor cells themselves, wherein the single cell clones express a neoantigen that is relevant to the subject.

According to one embodiment, the vaccine does not comprise cells of the tumor other than those of the single cell clone that has been found to express at least one neoantigen relevant to the subject.

According to another embodiment, the vaccine comprises cells of only two single cell clones, each being identified to express at least one neoantigen relevant to the subject.

According to another embodiment, the vaccine comprises cells of only three single cell clones, each being identified to express at least one neoantigen relevant to the subject.

According to another embodiment, the vaccine comprises cells of only four single cell clones, each being identified to express at least one neoantigen relevant to the subject.

According to another embodiment, the vaccine comprises cells of only five single cell clones, each being identified to express at least one neoantigen relevant to the subject.

According to another embodiment, the vaccine comprises cells of no more than 5 single cell clones, each being identified to express at least one neoantigen relevant to the subject.

According to another embodiment, the vaccine comprises cells of no more than 10 single cell clones, each being identified to express at least one neoantigen relevant to the subject.

The single cell clones which are present in the vaccine express at least one neoantigen relevant to the subject, at least two neoantigens relevant to the subject, at least three neoantigens relevant to the subject, at least four neoantigens relevant to the subject, at least five neoantigens relevant to the subject, at least six neoantigens relevant to the subject, at least seven neoantigens relevant to the subject, at least eight neoantigens relevant to the subject, at least nine neoantigens relevant to the subject, at least ten neoantigens relevant to the subject.

According to one embodiment of the present invention, the cells of the single cell clone are viable.

According to one embodiment of the present invention, the cells of the single cell clone are inactivated prior to administration.

As used herein, the phrase “inactivated tumor cells” refers to naïve tumor cell that have been rendered incapable of no more than three rounds of cell division to form progeny. The cells may nonetheless be capable of response to stimulus, or biosynthesis, antigen presentation, and/or secretion of cell products such as cytokines. Methods of inactivation are known in the art. Preferred methods of inactivation are treatment with toxins such as mitomycin C (preferably at least 10 μg/mL; more preferably at least about 50 μg/mL), or irradiation (preferably with at least about 5,000 cGy, more preferably at least about 10,000 cGy, more preferably at least about 20,000 cGy). Cells that have been fixed or permeabilized and are incapable of division are also examples of inactivated cells.

The present invention further contemplates vaccines comprising a combination of naïve, viable cells of the single cell clones and inactivated cells of the single cell clones.

In yet another embodiment, the cells of the single cell clone are genetically modified. Exemplary proteins, which may be expressed, include, but are not limited to interleukin 2 (IL-2), IL-8, IL-4, IL-6, gamma-interferon (IFN-γ), and granulocyte-macrophage colony stimulating factor (GMCSF).

Alternatively, the cells of the single cell clone are not genetically modified.

In still another embodiment, the cells are chemically activated to increase immunogenicity such as chemical modification with materials such as haptens or dinitrophenyl (DNP).

Alternatively, the cells of the single cell clone are not chemically activated.

The vaccines of the present invention may further comprise an adjuvant.

The term “adjuvant” as used herein refers to an agent that nonspecifically increases an immune response to a particular antigen thereby reducing the quantity of antigen necessary in any given vaccine and/or the frequency of injection necessary in order to generate an adequate immune response to the antigen of interest. Suitable adjuvants for use herein include, but are not limited to, poly IC; synthetic oligodeoxynucleotides (ODNs) with a CpG motif; modified polyinosinic:polycytidylic acid (Poly-IC) including, but not limited to, Poly-IC/LC (Hiltonol) and Poly-IC12U (Ampligen); Poly-K; carboxymethyl cellulose (CMC); Adjuvant 65 (containing peanut oil, mannide monooleate, an aluminum monostearate); Freund's complete or incomplete adjuvant; mineral gels such as aluminum hydroxide, aluminum phosphate, and alum; surfactants such as hexadecylamine, octadecylamine, lysolecithin, dimethyldioctadecylammonium bromide, N,N-dioctadecyl-N′,N″-bis(2-hydroxymethyl)propanediamine, methoxyhexadecylglyerol and pluronic polyols; polyanions such as pyran, dextran sulfate, polyacrylic acid, and carbopol; peptides such as muramyl dipeptide, dimethylglycine and tuftsin; and oil emulsions. The adjuvants of the present invention may include nucleic acids based on inosine and cytosine such as poly I:poly C; poly IC; poly dC; poly dI; poly dIC; Poly-IC/LC; Poly-K; and Poly-IC12U as well as oligodeoxynucleotides (ODNs) with a CpG motif, CMC and any other combinations of complementary double stranded IC sequences or chemically modified nucleic acids such as thiolated poly IC as described in U.S. Pat. Nos. 6,008,334; 3,679,654 and 3,725,545.

The peptide-based vaccines and/or cell based vaccines disclosed herein are capable of being used in combination with another therapeutic. Examples of therapeutics that can be used in conjunction with the vaccines disclosed herein include, but are not limited to: immunomodulatory cytokines, including but not limited to, IL-2, IL-15, IL-7, IL-21, GM-CSF as well as any other cytokines that are capable of further enhancing immune responses;

immunomodulatory antibodies, including but not limited to, anti-CTLA4, anti-CD40, anti-41BB, anti-OX40, anti-PD1 and anti-PDL1; and immunomodulatory drugs including, but not limited to, lenalidomide (Revlimid).

In addition, the peptide-based and/or cell based vaccines disclosed herein may be administered for cancer treatment in combination with chemotherapy in regimens that do not inhibit the immune system including, but not limited to, low dose cyclophosphamide and taxol. The vaccines may also be administered for cancer in combination with therapeutic antibodies including, but not limited to, anti-HER2/neu (Herceptin) and anti-CD20 (Rituxan).

Exemplary cancers that may be treated using the vaccines of the present invention include but are not limited to sarcomas (e.g., synovial sarcoma, osteogenic sarcoma, leiomyosarcoma uteri, and alveolar rhabdomyosarcoma), lymphomas (e.g., Hodgkin lymphoma and non-Hodgkin lymphoma), hepatocellular carcinoma, glioma, head-neck cancer, acute lymphocytic cancer, acute myeloid leukemia, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer (e.g., colon carcinoma), esophageal cancer, cervical cancer, gastrointestinal cancer (e.g., gastrointestinal carcinoid tumor), hypopharynx cancer, larynx cancer, liver cancer, lung cancer, malignant mesothelioma, melanoma, multiple myeloma, nasopharynx cancer, ovarian cancer, pancreatic cancer, peritoneum, omentum, and mesentery cancer, pharynx cancer, prostate cancer, rectal cancer, renal cancer, small intestine cancer, soft tissue cancer, stomach cancer, testicular cancer, thyroid cancer, ureter cancer, and urinary bladder cancer.

Various regimens of administration are contemplated by the present invention. For example, an exemplary regimen comprises vaccinating on at least separate occasions. According to one embodiment a period of time of about one week, two weeks, three weeks, four weeks or more is waited between each inoculation. The tumor cells and/or neoantigen of the present invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the tumor cells and/or neoantigens accountable for the biological effect.

Hereinafter, the phrases “physiologically acceptable carrier” and “pharmaceutically acceptable carrier” which may be interchangeably used refer to a carrier or a diluent that does not abrogate the biological activity and properties of the administered compound. The carrier may also include biological or chemical substances that modulate the immune response.

Techniques for formulation and administration of drugs may be found in “Remington's Pharmaceutical Sciences,” Mack Publishing Co., Easton, Pa., latest edition, which is incorporated herein by reference.

Suitable routes of administration include systemic delivery, including intramuscular, intradermic, subcutaneous, intravenous and intraperitoneal injections. Preferably, the tumor cells of the present invention are administered subcutaneously or intravenously.

In one embodiment, the pharmaceutical composition and the mode of delivery should be compatible with maintaining cell viability. Thus, the gauge of the syringe should be selected not to cause shearing and the pharmaceutical composition should not comprise any component toxic to cells etc.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer or inert growth medium.

The pharmaceutical composition described herein may be formulated for parenteral administration, e.g., by bolus injection or continuous infusion. Pharmaceutical compositions suitable for use in context of the present invention include compositions wherein the active ingredients are contained in an amount effective to achieve the intended purpose. More specifically, a therapeutically effective amount means an amount of active ingredients (e.g. tumor cells) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from animal studies. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide sufficient immune activation to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks months or years or until cure is effected or diminution of the disease state is achieved.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

The amount of a composition/vaccine to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above. The active ingredient may be prepared in such a way that it may be viably transferred to a distant location.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

GENERAL MATERIALS AND METHODS

Mice: Animals were maintained in a specific pathogen-free (SPF), temperature-controlled (22° C.±1° C.) mouse facility on a reverse 12-hour light, 12-hour dark cycle at the Weizmann Institute of Science. Food and water were given ad libitum. All mice studied were females on C57BL/6JO1aHsd background. C57/B6 animals were purchased from Harlan.

Cell lines: Murine melanoma B2905 and B 16F10.9 were used. The B2905 cell line was derived from a UV-irradiated HGF-transgenic mouse on a C57BL/6 background. B2905 were grown in RPMI containing 10% FCS, 1% L-glutamine, 1% PS antibiotics and 12.5 mM HEPES buffer. B 16F10.9 were grown in DMEM containing 10% FCS, 1% L-glutaime and 1% PS. Cells were grown in 10 cm plates, exposed to UVB using bench XX-15M 302 nm UV lamp and irradiation was measured using the UVX radiometer (Ultra Violet Products, Cambridge, UK). For in vitro proliferation assays, cells were grown in 96 wells plate (500 cells per well) and their proliferation was monitored daily using SyberGreen. For single clone generation, cells were plated in 96 well in concentration of 1 cell/well and grown for 14 days, and single clones were picked and expanded. For Western blot analysis, cells were harvested from 10 cm plates 24 hours post irradiation and immunoblots were performed. Lysates were stained using anti-mouse p53 (Cell signaling) and GAPDH (Sigma-Aldrich).

In vivo tumor inoculation: For experiments with the B2905 cell line, 5×10⁵ cells were injected in 100 μl PBS intradermally into the right lower flank after shaving. For experiments with the B 16F10.9 cell line, 10⁵ cells were injected. Tumors were measured using calipers. Tumor volume was assessed by measuring tumor diameter and calculation using the equation 0.5*(tumor diameter)³. Mice with tumor volume of >=1 were euthanized.

Flow cytometry: Tumors were surgically removed from animals and were place in cold PBS. Following, tumors were mechanically shredded using scalpel and incubated in RPMI medium containing 2 mg/ml Collagenase IV, 1 mg/ml Hyalurodinase and 2 mg/ml DNAseI (all Sigma-Aldrich) in room temp for two hours. The resulting cell suspension was filtered through a 70-μm mesh and cells were incubated in FACS buffer (PBS with 1% BSA, 2 mM EDTA and 0.05% sodium azide) in the presence of staining antibody. For intracellular staining, the CytoFix/Cytoperm Kit (BD) was used according to the manufacturer's instruction. Cells were acquired on FACSCanto, LSRII, and LSRFortessa systems (BD) and analyzed with FlowJo software (Tree Star). Antibodies used for flow cytometry were anti-mouse CD8 (clone 53-6.7), CD4 (clone GK1.5), TCRβ (clone H57-592), CD107a (clone 1D4B), CD137/41BB (clone 1765), Granzyme B (clone GB11), CD137 (clone 17B5) and IFN-γ (clone XMG12, all Biolegend). PE- labeled ASLTHVDSL (SEQ ID NO: 4) tetramer was obtained from the NIH tetramer core, and was incubated for 30 minutes in room temperature after staining of all additional antibodies. CellTracker violet (Molecular Probes) was used to stain proliferating TILs.

Histology: Mouse tumors derived B2905 cells were harvested at day 11 post inoculation, fixed in 4% (w/v) paraformaldehyde for 24 hours and restored in 1% paraformaldehyde until embedded in paraffin for histological analysis. In order to evaluate the distribution of T-cells lymphocytes, paraffin sections were double stained for CD8+ and CD3⁺ cells or CD4⁺ and CD3⁺ cells, and nuclear staining with 4′,6-Diamidino-2-phenylindole dihydrochloride (DAPI). Staining included the following antibodies: Rat anti-mouse CD8 (CN-14-9766-82 ebioscience), Rat anti-mouse CD4 (CN-14-0808-82 ebioscience), Rat anti-mouse CD3 (CN-MCA1477 SER).

Sequencing and genetic heterogeneity analysis: Genomic DNA was extracted from cell lines using the Qiagene blood mini kit Library Preparation and Sequencing Whole Exome sequencing libraries were prepared with Illumina-compatible SureSelectXT Library Prep Reagent Kit (Agilent Technologies, Santa Clara, Calif., USA) at the Genotypic Technology Pvt. Ltd., Bangalore, India. Briefly, 250 ng of genomic DNA (measured by Qubit fluorometer) was sheared by adaptive focused acoustics using a Covaris 5220 system (Covaris, Woburn, Mass., USA). The fragment size distribution (200 bp to 500 bp range) was verified on Agilent 2200 TapeStation with D1000 DNA screen tapes and reagents (Agilent Technologies, Palo Alto, Calif., USA). The fragments were endrepaired, adenylated and ligated to Illumina adaptors as per SureSelectXT library preparation kit protocol. The adapters used in the study were Illumina Universal Adapters: 5′ AATGATACGGCGACCACCGAGATCTACACTCTTTCCCTACACGACGCTCTTCCG ATCT-3′ (SEQ ID NO: 1) and Index Adapter: 5′-GATCGGAAGAGCACACGTCTGAACTCCAGTCAC [SEQ ID NO: 2, INDEX] ATCTCGTATGCCGTCTTCTGCTTG-3′ (SEQ ID NO: 3). The adapter-ligated DNA was purified by HighPrep magnetic beads and then amplified for 10 cycles of PCR using Illumina-compatible primers provided in the SureSelectXT kit. The amplified fragments were purified by HighPrep beads and the concentration was measured by Qubit fluorometer. The fragment size was again checked on Agilent 2200 TapeStation with D1000 DNA screen tapes. Target enrichment was performed according to the manufacturer's instructions using SureSelect Mouse all exon capture baits. In-solution hybridization was performed for 20 hours at 65° C. After hybridization, the captured targets were pulled down by biotinylated probe-target hybrids using streptavidin-coated magnetic beads (Dynabeads MyOne Streptavidin T1, ThermoFisher scientific Inc.). The magnetic beads were washed according to the manufacturer's instructions and resuspended in 15 μl of nuclease free water. The captured DNA libraries were amplified by PCR including appropriate indexing primer for each sample. The final PCR product (sequencing library) was purified with HighPrep beads, followed by quantification by Qubit fluorometer (Thermo Fisher Scientific, Mass., USA) and fragment size distribution was analyzed on Agilent 2200 TapeStation (see appendix). Finally, the sequencing libraries were pooled in equimolar amounts to create a final multiplexed library pool for sequencing on an Illumina sequencer for 150 bp paired-end chemistry.

Analysis of sequencing: Whole-exome sequencing reads were de-duplicated and aligned to the mouse genome assembly GRCm38 [Cibulskis, K., et al. Sensitive detection of somatic point mutations in impure and heterogeneous cancer samples. Nature biotechnology 31, 213-219 (2013)] without normal sample. VCF files were converted to MAF files (v2.4) using vcf2maf. Called exonic mutations and the tri-nucleotide 96 mutations context were counted to assess the genomic effect of UVB treatment in the mutational landscape of the cell lines. To evaluate the genetic heterogeneity of the cell lines, we look at the variant allele frequency (VAF) of the exonic mutations and defined mutations with a VAF>0.25 as more likely to be clonal mutations and vice versa, as previously defined [Williams, M. J., Werner, B., Barnes, C. P., Graham, T. A. & Sottoriva, A. Identification of neutral tumor evolution across cancer types. Nature genetics 48, 238-244 (2016)].

Production and purification of membrane HLA molecules: B2905 cells were grown at 500×10⁶ in triplicates and were pelleted. For B16F10.9 cells, 300 U/ml of murine interferon-γ was administrated 24 h before pelleting. Cells lysate from two B2905 cell lines were used for immunoaffinity purification of MHC molecules with their bound peptides, using the 20-8-4 and 28-14-8 antibodies against H2-Kb and H2-Db respectively covalently bound to Protein-A Sepharose beads The HLA peptides were recovered from HLA molecules with 1% TFA followed by separation of the peptides from the proteins contaminants by binding the eluted fraction to disposable reversed-phase C18 columns (Harvard Apparatus). Elution of the peptides was done with 30% acetonitrile and 1% TFA. The eluted peptides were cleaned also by C18 stage tip.

Identification of the MHC peptides: The HLA peptides were dried by vacuum centrifugation, re-solubilized with 0.1% Formic acid and resolved on capillary reversed phase chromatography on 0.075×200 mm laser-pulled capillaries, self-packed with 3μ Reprosil-Aqua C₁₈. Electrospray tandem mass spectrometry was performed with the Q-Exactive-Plus mass spectrometer (Thermo Scientific). The MS data was analyzed by MaxQuant version 1.5.0.25, with 5% FDR. Peptide identifications were based on the mouse section of the Uniprot database from May 2016 combined with the wild type and mutant protein sequences.

Ex vivo activation assay: Spleen and bone marrow were extracted from naïve C57/B6 mouse. Splenocytes were plated in 6 wells pre-coated with CD28 (clone CD28.2) and CD3 (clone HIT39) and grown in B2905 media supplemented with 50 Um β-mercaptoethanol and 10U/ml IL-2 for 2 days. Cells were harvested and re-plated with IL-2 and kept for 6 days. In parallel, bone marrow derived cells were grown in the presence of GM-CSF (10 ug/ml, Peprotech) for 6 days. The resulting bone-marrow derived DCs were plated in 24 wells and incubated 10 ug with designated peptides which were dissolved in DMSO (1% DMSO in PBS) for 24 hours. Peptides were washed and splenic T cells were added and co-cultured with DCs for 24 hours. The suspension was taken for FACS analysis.

### Example 1

### A Reduction in Intra-Tumor Heterogeneity (ITH) Enhances Immune Rejection

### RESULTS

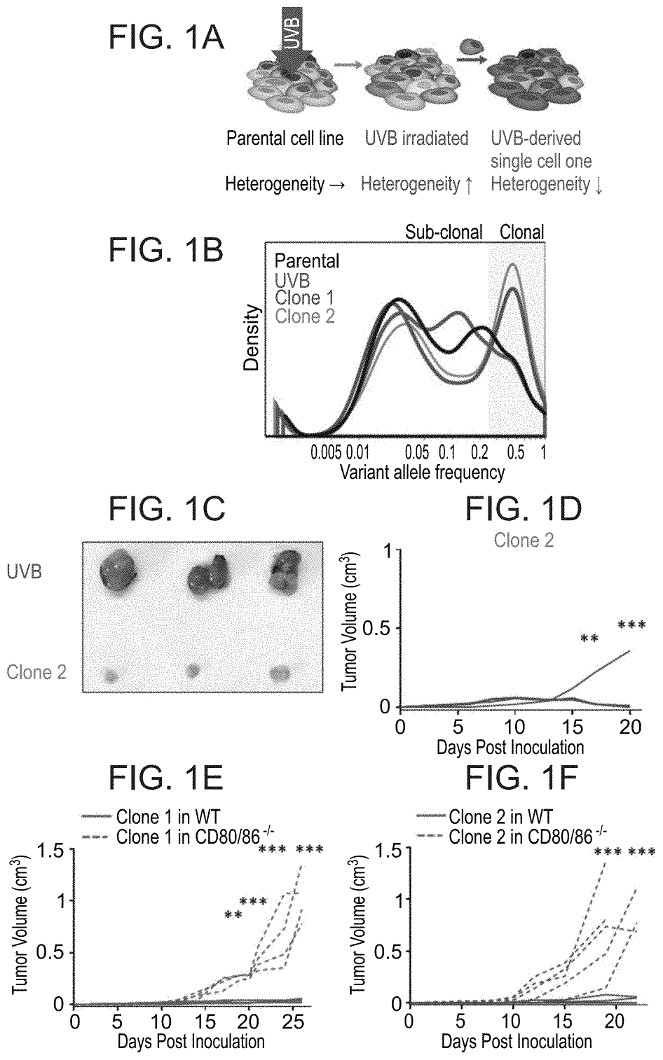

Single cell clones were generated from irradiated B2905 cells (FIG. 1A). Unlike the increased tumor growth seen in the parental UVB-irradiated parental cells, the two B2905 irradiated single cell clones grew at a dramatically reduced rate in vivo; clone 1 hardly generated any tumors, and clone 2 gave rise to small-size tumors that were spontaneously rejected (FIG. 1B-D). However, when inoculated into CD80/86^{-/-} double knock-out mice that are devoid of appropriate T-cell stimulation, both clones grew considerably more aggressively than in the wild-type mice, testifying that they are indeed tumorigenic in the absence of a T-cell response (FIG. 1E-F). Correspondingly, a similar single cell clone derived from UVB-exposed B 16F10.9 cells, designated B16 clone 1, was found to be considerably less tumorigenic; clone 1 hardly grew any tumors in WT mice, and grew highly aggressive tumors in CD80/86^{-/-} mice (FIGS. 2A-B). The present inventors then postulated that single cell clones derived from the parental, non-irradiated cell line, might also undergo such immune rejection due to their homogeneity. Indeed, non UVB clones 1 and 2 grew considerably less than the parental cell line in vivo, similar to the growth of the UVB vs. UVB-derived single cell clones. Thus, tumors with low ITH grow to a much lesser extent in immunocompetent mice, regardless of their mutational load or UVB irradiation.

### Example 2

### Detection of Reactive Neoantigens in Single Cell Derived Clones Using HLA Peptidomics

To further study the role of reduced tumor cell heterogeneity on the presentation of immunogenic neo-antigens, HLA peptidomics was employed to identify tumor-specific neo-antigens in the various B2905-established cells lines. This approach entailed purifying MHC molecules by immunoaffinity and then analyzing the MHC-eluted peptides by capillary chromatography and tandem mass spectrometry (MS). The MS spectra were queried against the mouse proteome dataset (Methods), to which the present inventors added the peptides inferred from the mutations identified by whole exome sequencing (WES). No neo-antigens were detected in the parental irradiated and non-irradiated cells; however, two neo-antigens (ASLTHVDSL (SEQ ID NO: 4), encoded by IFI44_{L>S} and presented on the H2-Db allele, and SAYEKLYSL (SEQ ID NO: 5), encoded by RPTOR_{H>Y} and presented on the H2-K_{b} allele) were identified in clone 2 only. Synthetic peptides had similar mass spectra, indicating appropriate identification. In the B16F10.9 cell line, a potential neo-antigen which was shared in both parental, UVB irradiated and clone 1 (ANFIFRQL (SEQ ID NO: 6), encoded by Pask_{S>N} presented by the H2-K_{b} allele) was identified by HLA peptidomics as well as a potential neo-antigen which was specific to clone 1 only (TIYVFPERL (SEQ ID NO: 7), encoded by Mlkl_{S>F}, also presented by the H2-K_{b} allele). Thus, the presence of single clone-specific neo-antigens might facilitate anti-tumor response.

To test the reactivity of the B2905 peptides, autologous bone marrow derived dendritic cells (DCs) were prepared and loaded with 10 ug of either WT or mutant peptides. The cells were co-cultured for 24 hours with spleen-derived effector T-cells taken from naïve C57/B6 mice and grown ex vivo, and then T-cell reactivity was measured by evaluating the expression of cell surface marker CD137/41BB. The strongest and most specific reactivity was towards mutant peptide IFI44_{L>S}, thus indicating the pre-determined presence of neo-antigen-specific clones within the T-cell repertoire of the mouse. Using tetramer staining of the ASLTHVDSL (SEQ ID NO: 4) and SAYEKLYSL (SEQ ID NO: 5) bound to MHC, CD8+ TILs specific for this peptide were found in both parental and clone 2 derived tumors, whereas in UVB derived tumors the neo-antigen specific TIL fractions were reduced by ˜80% for both neoantigen specific TILs.

To conclude, the detection of neo-antigens and their specific TILs, the presence of unique and oligoclonal TCR repertoire in TILs populating single clone-derived tumors, the successful infiltration into the tumor core, and the robust rejection response seen in vivo, suggest that single clone-derived tumors are rejected due to the more efficient immune response that they elicit.

### Example 3

Tumors Derived from Mixtures of Clones Elicit Diminished Anti-Tumor Response

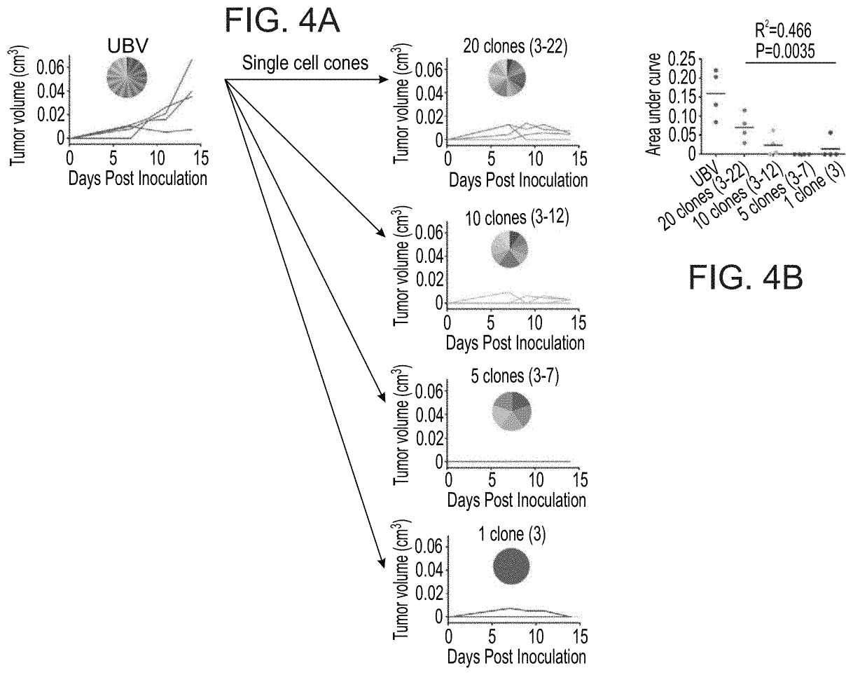

The present results suggest that in the extreme scenario of tumors dominated by a single clone, the immune system can home easily on the tumor cells and eliminate them. The present inventors then postulated that less extreme scenarios in which they would control experimentally the degree of heterogeneity by mixing different single cell clones in different compositions. New single cell clones, designated 3 to 22, were generated from the UVB irradiated B2905 cells, and 5, 10 and 20 clones were mixed in even amounts and administrated to mice (total amounts of cells injected were always 5*10⁵ per mouse; FIG. 4A). First, they tested the ability of clones 3-22 and their mixes to grow in vitro. The various clones demonstrated varying degree of in vitro growth; however, when grown in mixes of 5, 10 and 20 clones, the overall growth of the clones was similar, indicating that no single clone was able to take over the total growth (FIG. 4B). They then tested each individual clone and the various mixes in vivo. Strikingly, all the clones grew to much lesser extent than the original, UVB irradiated B2905 cell line, and all of the 20 clones were eventually rejected, indicating that the immunorejection seen in clones 1 and 2 was the rule, rather than an exception. 20% (4/20) of the clones did not form any tumors, and the remaining 80% were all rejected, with overall mean day of average being day 8.5. The 5 and 10 clone mixes were rejected similarly (average day of rejection 10.333 and 7.333 respectively). However, the 20 clone mix was at average day 20.333, which was later from both 5 and 10 mixes, as well as 95% (19/20) of the individual clones. Tumor growth of the clones and their mixes mirrored the finding of later rejection of the 20 clone mix. Thus, increasing tumor heterogeneity artificially by mixing >10 clones diminished anti-tumor immunity.

### Example 4

### Single Cell Clone Analysis, Phylogenic Tree Construction, Determination of Vaccination Efficiency, and NeoA Discovery

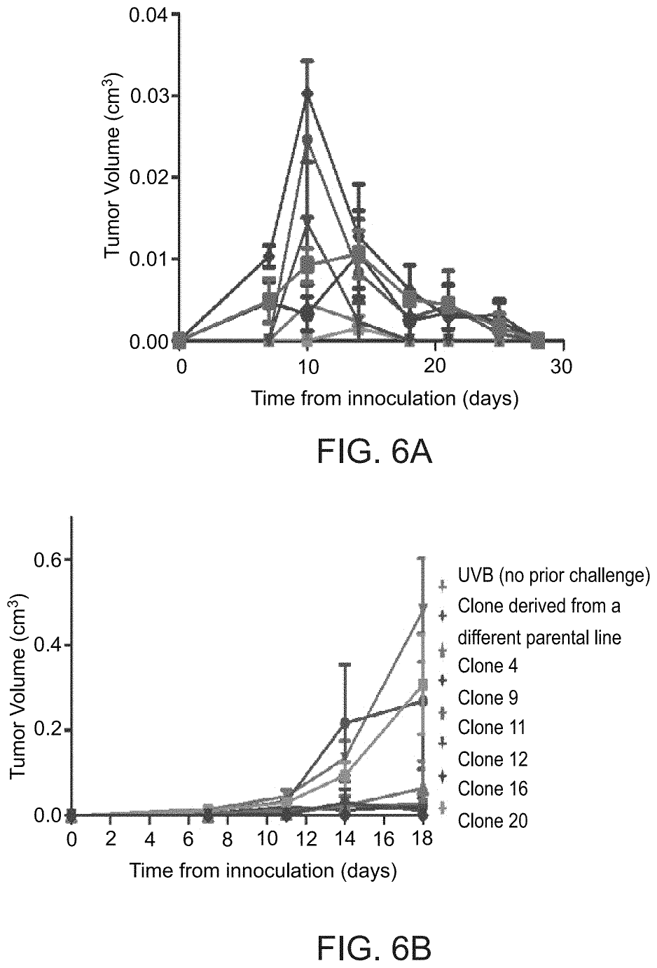

To derive the subclonal architecture of the UVB-irradiated cell line, the present inventors reanalyzed its whole exome sequence (WES), as well as 20 single clones derived from it, and inferred a phylogenetic tree with six branches (FIGS. 5A-B). This allowed not only the reconstruction of the tumor's clonal evolution, but also its utilization for rational vaccination modalities by identifying clones that harbor clonal mutations, shared by all tumor cells, found in the stem of the tree, or, alternatively, by using mixtures of clones found across different branches. Importantly, when they inoculated the highly aggressive UVB-irradiated cell line (containing high ITH) into mice that were previously injected with single cell lines (low ITH) that were rejected, in 5/6 (83%) of the cases, the UVB line did not form tumors (FIGS. 6A-B). This intriguing result demonstrates the potential that a single clone derived from the parental tumor has in mediating a protective anti-tumor effect.

### Example 5

### Establish Human AutoCCVs Catered to ITH, the Tumor Phylogenetic Tree and T Cell Reactivity

Cancer is an evolutionary process characterized by the accumulation of somatic mutations in a population of cells that can be phylogenetically reconstructed as the life history of a given cancer. The trunk of the phylogenetic tree describes events present in all cancer cells derived from the cell of origin and are, therefore, clonal. Branched events are subclonal mutations that occur in a subset of the progeny. Targeting immunogenic truncal mutations may maximize tumor response, emphasizing the importance of accurately determining the tumor's clonal events and assessing their immunogenicity.

In order to test if the ex-vivo system described herein can identify clonal immunogenic clones that enhance the delayed-type hypersensitivity (DTH) response and increase relapse-free survival, new stage III and resectable stage IV melanoma patients who will be vaccinated with their autologous tumor will be prospectively studied. Whole exome sequence (WES) will be performed on the original tumor, its derived cell line, as well as on the patient's blood, for somatic mutation analysis. Single cell clones will be established from the cell lines and WES will be performed in order to derive a human phylogenetic tree on the primary tumor, as well as to identify presented NeoAs. Based on the ITH level of the primary tumor and the phylogenetic tree structure, particular single cell clones will be assessed for immunogenicity using a co-culture with PBMC, as well as PBMC proliferation and intracellular staining of IFNγ. These experiments can be assessed in HTP format using an automated imager and flow cytometry. Single cell clones that induce T cell expansion and IFNγ production will be considered immunogenic.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of identifying a neoantigen in a tumor of a subject comprising:
(a) culturing cells of the tumor under conditions which generate single cell clones of said tumor; and
(b) analyzing said single cell clones for expression of a neoantigen which is reactive with T cells from said subject, thereby identifying the neoantigen in a tumor of a subject.

2. The method of claim 1, wherein said cells of the tumor comprise cells of a solid tumor.

3. The method of claim 1 or 2, wherein said cells of the tumor comprise melanocytes.

4. A method of preparing a vaccine for treating a tumor of a subject comprising:
(a) culturing cells of the tumor under conditions which generate single cell clones of said tumor; and
(b) analyzing said single cell clones for expression of a neoantigen which is reactive with T cells from said subject; and
(c) preparing a vaccine which comprises said neoantigen.

5. The method of claim 1 or 4, wherein said culturing comprises passaging said cells of the tumor for no more than 20 passages.

6. The method of claim 5, wherein said culturing comprises passaging said cells of the tumor for no more than 10 passages.

7. The method of claim 4, wherein said vaccine comprises a single cell clone which expresses said neoantigen.

8. The method of claim 7, wherein a variant allele frequency (VAF) of the exonic mutations of cells of said single cell clone is greater than 0.25.

9. The method of claim 7, wherein a variant allele frequency (VAF) of the exonic mutations of cells of said single cell clone is greater than 0.65.

10. The method of claim 7, wherein said vaccine comprises no more than ten single cell clones.

11. The method of claim 4, wherein said vaccine comprises dendritic cells which present said neoantigen.

12. The method of claim 1 or 4, wherein said cells of the tumor comprise cells of a solid tumor.

13. The method of any one of claim 1, 4 or 12, wherein said cells of the tumor comprises melanoma cells.

14. A vaccine comprising cells of a single tumor cell clone, said single tumor cell clone expressing at least one neoantigen, wherein a variant allele frequency (VAF) of the exonic mutations of said cells is greater than 0.25.

15. The vaccine of claim 14, wherein a variant allele frequency (VAF) of the exonic mutations of cells of said single cell clone is greater than 0.65.

16. The vaccine of claim 14, wherein said single tumor cell clone comprises primary cells.

17. The vaccine of claim 14 or 16, wherein said cells are viable.

18. The vaccine of claim 14 or 16, wherein said cells are irradiated.

19. The vaccine of claim 14, further comprising an adjuvant.

20. The vaccine of claim 14, wherein said single tumor cell clone has been passaged for no more than 20 passages.

21. The vaccine of any one of claims 14-20, wherein said tumor cell clone comprises melanocytes.

22. A method of treating a cancer of a subject comprising:
(a) preparing a vaccine according to any one of claims 4-13; and
(b) vaccinating said subject with said vaccine, thereby treating the cancer of the subject.

23. The vaccine of any one of claims 14-21, for use in treating a cancer.
